(19) United States
(12) Patent Application Publication
(10) Pub. No.: US 2012/0195837 A1
(43) Pub. Date: Aug. 2, 2012

(54) TIN(II)-2-HYDROXYCARBOXYLATES

(75) Inventors: CHRISTIAN WERNER, Hannover (DE); CHRISTOPH STEINBERG, Garbsen (DE); GIUSEPPE GROTTI, HEVERLEE (BE); BJOERN JACKISCH, Isernhagen (DE)

(73) Assignee: HONEYWELL INTERNATIONAL INC., MORRISTOWN, NJ (US)

(21) Appl. No.: 13/326,755

(22) Filed: Dec. 15, 2011

Related U.S. Application Data
(60) Provisional application No. 61/437,180, filed on Jan. 28, 2011.

(51) Int. Cl. C07F 7/22; A61Q 11/00; C08G 18/16; A61K 8/58

(52) U.S. Cl. 424/55; 556/105; 528/58

(57) ABSTRACT

The invention is drawn to a composition comprising a tin(II) 2-hydroxycarboxylate, where the 2-hydroxycarboxylate is preferably selected from the group consisting of glycolate, lactate, mandelate and 2-hydroxybutyrate. The tin(II) 2-hydroxycarboxylates may be prepared by a method comprising the steps of (a) providing an aqueous solution of a tin(II) salt, (b) adding a 2-hydroxycarboxylic acid, or a salt of a 2-hydroxycarboxylic acid, (c) further adding a base, wherein the tin(II) 2-hydroxycarboxylate precipitates, (d) optionally, isolating the tin(II) 2-hydroxycarboxylate, and (e) optionally, drying. The tin(II) 2-hydroxycarboxylates and compositions containing them are useful in oral care applications, and as catalysts for chemical condensation reactions and/or the production of polymers.

### CROSS-REFERENCE TO RELATED APPLICATIONS

The present application claims the benefit of priority under 35 U.S.C. §119(e) of U.S. Provisional Application Ser. No. 61/437,180, filed on Jan. 28, 2011, the entire disclosure of which is incorporated by reference herein.

### FIELD OF THE INVENTION

The invention is related to the field of tin(II) chemical reagents, their preparation, and their use in oral care applications and as chemical reaction catalysts.

### BACKGROUND OF THE INVENTION

Stannous chloride dihydrate (SnCl₂.2H₂O) is a widely used source of stannous, or tin(II), ion in various industries, including the chemical industry and the personal care industry. However, this compound has a number of physical properties that make it difficult to store as well as to handle, transport and use. The commercial solid material tends to cake. It is also highly corrosive to typical industrial equipment, posing problems for storage and transport, as well as for use as a reactant in various chemical processes.

### BRIEF SUMMARY OF THE INVENTION

The invention is directed to the discovery that tin(II) salts of 2-hydroxycarboxyic acids, for example, tin(II) lactate, show dramatically improved physical properties, versus stannous chloride dihydrate. Thus, tin(II) lactate is observed to be a non-hygroscopic, and therefore an easily handlable free-flowing powder. In addition, tin(II) lactate is observed to be non-corrosive.

One embodiment of the invention is drawn to a composition comprising a tin(II) 2-hydroxycarboxylate. Preferred 2-hydroxycarboxyic acids are selected from the group consisting of glycolic acid, lactic acid, mandelic acid and 2-hydroxybutyric acid. Most preferred is the composition comprising tin(II) lactate.

Another embodiment of the invention is directed to a method of preparing a tin(II) 2-hydroxycarboxylate, comprising the steps of:

(a) providing an aqueous solution of a tin(II) salt,

(b) adding a 2-hydroxycarboxylic acid or a salt of a 2-hydroxycarboxylic acid, or a solution thereof,

(c) further adding a base to adjust the pH of the solution, wherein the tin(II) 2-hydroxycarboxylate precipitates,

(d) optionally, isolating said tin(II) 2-hydroxycarboxylate, and

(e) optionally, drying.

Alternatively an aqueous solution of a tin(II) salt may be added to a solution of a 2-hydroxycarboxylic acid or a salt of a 2-hydroxycarboxylic acid, that is, steps (a) and (b) may be reversed.

A further embodiment of the invention is drawn to a method of oral care, comprising the steps of (a) incorporating the above composition into an oral care formulation to form a tin(II)-containing oral care preparation, and (b) applying said oral care preparation to the mouth, teeth and/or gums.

Yet another embodiment of the invention is directed to a method of chemical condensation reaction comprising the step of adding a composition comprising a tin(II) 2-hydroxycarboxylate as a catalyst. Analogously, still another embodiment of the invention is drawn to a method of forming a polymer comprising the step of adding a composition comprising a tin(II) 2-hydroxycarboxylate as a catalyst to a monomer mixture.

### DETAILED DESCRIPTION OF PREFERRED EMBODIMENTS

Applicants have discovered that tin(II) salts of 2-hydroxycarboxyic acids, for example, tin(II) lactate, show dramatically improved physical and chemical properties, versus stannous chloride dihydrate. For example, tin(II) lactate is observed to be a non-hygroscopic, free-flowing powder. In addition, tin(II) lactate is observed to be non-corrosive toward standard industrial equipment.

One embodiment of the invention is drawn to a composition comprising a tin(II) 2-hydroxycarboxylate. The preferred 2-hydroxycarboxyic acids are selected from the group consisting of glycolic acid, lactic acid, mandelic acid and 2-hydroxybutyric acid. These 2-hydroxycarboxyic acids can be chiral, as for example, the naturally occurring L-lactic acid, or they can be racemic. Most preferred is the composition comprising tin(II) lactate. A particularly preferred composition comprises tin(II) lactate derived from L-lactic acid.

The tin(II) 2-hydroxycarboxylates of the invention may be prepared via a process comprising the steps of:

(a) providing an aqueous solution of a tin(II) salt, preferably stannous chloride and/or stannous chloride dihydrate,

(b) adding a 2-hydroxycarboxylic acid, preferably glycolic acid, lactic acid, mandelic acid or 2-hydroxybutyric acid, or a salt of a 2-hydroxycarboxylic acid, such as sodium glycolate or sodium lactate; the 2-hydroxycarboxylic acid or salt thereof may be in the form of an aqueous solution, and if necessary, an organic cosolvent can be added, such as methanol, ethanol, isopropanol or acetone,

(c) further adding a base, for example, sodium hydroxide, potassium hydroxide, ammonium hydroxide, sodium bicarbonate, or sodium carbonate, in order to adjust the pH of the solution, wherein the tin(II) 2-hydroxycarboxylate precipitates,

(d) optionally, isolating said tin(II) 2-hydroxycarboxylate, preferably by filtration, and

(e) optionally, drying the tin(II) 2-hydroxycarboxylate, preferably under vacuum with heat, at a temperature up to 120° C., preferably up to 100° C., most preferably at a temperature in the range of 60-100° C.

Alternatively, the tin(II) 2-hydroxycarboxylates of the invention may be prepared via a process comprising the steps of:

(a) providing a solution of a 2-hydroxycarboxylic acid, preferably glycolic acid, lactic acid, mandelic acid or 2-hydroxybutyric acid, or a salt of a 2-hydroxycarboxylic acid, such as sodium glycolate or sodium lactate; preferably the solution is aqueous, and if necessary, an organic cosolvent can be added, such as methanol, ethanol, isopropanol or acetone,

(b) adding an aqueous solution of a tin(II) salt, preferably stannous chloride and/or stannous chloride dihydrate,

(c) further adding a base, for example, sodium hydroxide, potassium hydroxide, ammonium hydroxide, sodium bicarbonate, or sodium carbonate, in order to adjust the pH of the solution, wherein the tin(II) 2-hydroxycarboxylate precipitates,

(d) optionally, isolating said tin(II) 2-hydroxycarboxylate, preferably by filtration, and

(e) optionally, drying the tin(II) 2-hydroxycarboxylate, preferably under vacuum with heat, at a temperature up to 120° C., preferably up to 100° C., most preferably at a temperature in the range of 60-100° C.

A further embodiment of the invention is drawn to a method of oral care, comprising the steps of (a) incorporating a tin(II) 2-hydroxycarboxylate, or a composition comprising a tin(II) 2-hydroxycarboxylate, into an oral care formulation, for example, and without limitation, an oral dentifrice or oral wash, to form a tin(II)-containing oral care preparation, and (b) applying said oral care preparation to the mouth, teeth and/or gums, preferably applying to the teeth and/or gums. The composition, or tin(II) 2-hydroxycarboxylate, used in the method of oral care preferably comprises tin(II) lactate.

Optionally the method of oral care may further comprise a fluoride source in the oral care formulation, for example, and without limitation, sodium fluoride or sodium monofluorophosphate.

Application of the formulated tin(II) 2-hydroxycarboxylate to mouth, teeth and/or gums is typically for about 2 to about 30 minutes, preferably about 5 to about 10 minutes.

Following application of the oral care preparation, tin(II) is retained on the oral surfaces for a prolonged period of time, which provides an inhibiting action on plaque formation and on dental caries.

Yet another embodiment of the invention is directed to a method of chemical condensation reaction comprising the step of adding a tin(II) 2-hydroxycarboxylate, or a composition comprising a tin(II) 2-hydroxycarboxylate, as a catalyst. Depending on the nature of the reaction, the catalyst is added in amounts of about 0.0001 to about 10 mole %, preferably about 0.001 to about 1 mole %, or alternatively in amounts of about 0.001 to about 5 weight %, preferably 0.001 to about 1 weight %, based on the reactants, optionally dissolved in a suitable solvent.

Analogously, a further embodiment of the invention is drawn to a method of forming a polymer comprising the step of adding to a monomer mixture, optionally dissolved in a suitable solvent, a composition comprising a tin(II) 2-hydroxycarboxylate as a polymerization catalyst. The catalyst is added in amounts of about 0.0001 to about 10 mole %, preferably about 0.001 to about 1 mole %, or alternatively in amounts of about 0.001 to about 5 weight %, preferably about 0.001 to about 1 weight %. The reaction mixture is typically heated to about 60 to about 120° C., for about 1-12 hours.

As is commonly understood in the chemical arts, the term “stannous” refers to tin in the +2 oxidation state, or “tin(II)”. Also, as commonly understood in the chemical arts, in a chemical condensation reaction, two organic molecules unite, usually with the loss of water or other small molecule.

The invention may be further understood by means of the following Examples, which are not intended to be limiting, but rather illustrative of the preferred embodiments as presently known to Applicants.

### EXAMPLES

### Example 1. Preparation of Tin(II) Lactate

To 172.5 g of lactic acid in 100 g of water, a solution of SnCl₂ (337.0 g, SnCl₂ content 47.8%) is added at 60 ° C. 217 g of NaOH solution (32%) is added over 50 minutes while cooling the reaction mixture. The white suspension is stirred for 30 minutes, and the tin(II) lactate is separated by vacuum filtration. The solid product is washed with 400 mL of distilled water, and dried under vacuum at 60 ° C. for 16 h.

Yield: 163.5 g; Sn content is 57%.

### Example 2. Preparation of Tin(II) L-lactate

The procedure of Example 1 is repeated using L-lactic acid.

### Example 3. Use of Tin(II) 2-hydroxycarboxylates in Oral Care

A sample of tin(II) L-lactate (Example 2) is formulated as an oral dentifrice or oral wash, using additives and methods as known in the art. Application of the formulated tin(II) L-lactate to the mouth, teeth and/or gums for about 5 to about 10 minutes successfully results in retention of Sn(II) ion on oral surfaces. The application of tin(II) L-lactate reduces plaque formation and/or dental caries compared with a control group which lacks the Sn(II) application.

### Example 4. Use of Tin(II) 2-hydroxycarboxylates as Catalysts in Chemical Reactions

Tin(II) lactate (Example 1) is added in a catalytic amount (0.001 to 1 mole %) to a monomer mixture comprising polyisocyanates and polyols in a suitable solvent, and the reaction mixture is heated to about 60 to about 120° C. for about 1 to about 12 hours. The precipitated polyurethane is removed by filtration and dried in vacuo at about 60 to about 100° C.

### Example 5. Use of Tin(II) L-lactate as a Catalyst for Polymerization

The procedure of Example 4 is repeated with tin(II) L-lactate (Example 2).

What is claimed is:

1. A composition comprising a tin(II) 2-hydroxycarboxylate.

2. The composition of claim 1, wherein said 2-hydroxycarboxylate is selected from the group consisting of glycolate, lactate, mandelate, and 2-hydroxybutyrate.

3. The composition of claim 2, wherein said 2-hydroxycarboxylate comprises lactate.

4. A method of preparing a tin(II) 2-hydroxycarboxylate, comprising the steps of:
(a) providing an aqueous solution of a tin(II) salt,
(b) adding a 2-hydroxycarboxylic acid or a salt of a 2-hydroxycarboxylic acid, or a solution thereof,
(c) further adding a base, wherein the tin(II) 2-hydroxycarboxylate precipitates,
(d) optionally, isolating said tin(II) 2-hydroxycarboxylate, and
(e) optionally, drying.

5. The method of claim 4, wherein said tin(II) salt comprises SnCl2, optionally as a hydrate.

6. The method of claim 4, wherein said 2-hydroxycarboxylic acid comprises lactic acid.

7. The method of claim 4, wherein said tin(II) 2-hydroxycarboxylate is isolated by filtration.

8. The method of claim 4, wherein said tin(II) 2-hydroxycarboxylate is dried under vacuum.

9. A method of oral care, comprising the steps of:
(a) incorporating the composition of claim 1 into an oral care formulation to form a tin(II)-containing oral care preparation, and
(b) applying said oral care preparation to the mouth, teeth and/or gums.

10. The method of claim 9, wherein said composition comprises tin(II) lactate.

11. The method of claim 9, wherein the application is to the teeth and/or gums.

12. A method of chemical condensation reaction and/or polymer formation, comprising the step of adding a catalytically effective amount of the composition of claim 1 as a catalyst.

13. The method of claim 12, wherein said composition comprises tin(II) lactate.

14. A method of preparing a tin(II) 2-hydroxycarboxylate, comprising the steps of:
(a) providing a solution of a 2-hydroxycarboxylic acid or a salt of a 2-hydroxycarboxylic acid,
(b) adding an aqueous solution of a tin(II) salt,
(c) further adding a base, wherein the tin(II) 2-hydroxycarboxylate precipitates,
(d) optionally, isolating said tin(II) 2-hydroxycarboxylate, and
(e) optionally, drying.

15. The composition of claim 2, wherein said 2-hydroxycarboxylate comprises L-lactate.

16. The method of claim 4, wherein said 2-hydroxycarboxylic acid comprises L-lactic acid.

17. The method of claim 9, wherein said composition comprises tin(II) L-lactate.

18. The method of claim 12, wherein said composition comprises tin(II) L-lactate.

19. The method of claim 14, wherein said 2-hydroxycarboxylic acid comprises lactic acid.

20. The method of claim 14, wherein said 2-hydroxycarboxylic acid comprises L-lactic acid.
